(19) United States
(12) Patent Application Publication
(10) Pub. No.: US 2024/0416074 A1
(43) Pub. Date: Dec. 19, 2024

(54) Antimicrobial Catheters and Methods

(71) Applicant: Bard Access Systems, Inc., Salt Lake City, UT (US)

(72) Inventor: Chase Provost, West Bountiful, UT (US)

(21) Appl. No.: 18/209,379

(22) Filed: Jun. 13, 2023

(51) Int. Cl. A61M 25/00

(52) U.S. Cl. CPC A61M 25/0043; A61M 25/0014; A61M 2025/0056

(57) ABSTRACT

An antimicrobial catheter can include one or more polymeric inserts of a filled polymer in a catheter tube, a catheter hub, one or more extension legs, one or more extension-leg fittings or a combination thereof. Each polymeric insert of the one-or-more polymeric inserts can provide a portion of a luminal surface in a catheter component selected from the catheter tube, the catheter hub, the one-or-more extension legs, and the one-or-more extension-leg fittings. Further, each polymeric insert of the one-or-more polymeric inserts can elute one or more antimicrobial metal species therefrom upon contact with a liquid. A method of making such an antimicrobial catheter can include a polymeric insert-molding operation of molding a plurality of the polymeric inserts as well as a catheter component-molding operation of molding a plurality of at least one catheter component selected from the catheter tube, the catheter hub, an extension leg, and an extension-leg fitting.

### BACKGROUND

Catheter-related bloodstream infections (“CRBSIs”) are among the most frequent infections acquired in hospitals. The incidence of CRBSIs varies considerably depending on type of catheter, insertion site, clinician experience in catheter placement, duration of catheter placement, frequency of catheter access, and the like. While there has been a remarkable increase in epidemiological knowledge of CRBSIs as well as methodologies for diagnosis, management, and prevention thereof, CRBSIs still contribute to significant morbidity as well as lengthy and expensive hospital stays.

Disclosed herein are antimicrobial catheters and methods that address the foregoing.

### SUMMARY

Disclosed herein is an antimicrobial catheter including, in some embodiments, a catheter tube, a catheter hub, one or more extension legs, one or more extension-leg fittings, and one or more polymeric inserts of a filled polymer. A proximal-end portion of the catheter tube is coupled to the catheter hub, a distal-end portion of each extension leg of the one-or-more extension legs is coupled to the catheter hub, and a proximal-end portion of each extension leg of the one-or-more extension legs is coupled to an extension-leg fitting of the one-or-more extension-leg fittings. Each polymeric insert of the one-or-more polymeric inserts provides at least a portion of a luminal surface in a catheter component selected from the catheter tube, the catheter hub, the one-or-more extension legs, and the one-or-more extension-leg fittings. Further, each polymeric insert of the one-or-more polymeric inserts is configured to elute one or more antimicrobial metal species therefrom upon contact with a liquid.

In some embodiments, the catheter tube includes at least one polymeric insert of the one-or-more polymeric inserts.

In some embodiments, the catheter tube includes an antimicrobial compounded into the catheter tube different than the one-or-more antimicrobial metal species of the one-or-more polymeric inserts.

In some embodiments, the catheter hub includes at least one polymeric insert of the one-or-more polymeric inserts.

In some embodiments, each extension leg of the one-or-more extension legs includes at least one polymeric insert of the one-or-more polymeric inserts.

In some embodiments, each extension-leg fitting of the one-or-more extension-leg fittings includes at least one polymeric insert of the one-or-more polymeric inserts.

In some embodiments, the filled polymer of a polymeric insert includes a same base polymer as that of the catheter component including the polymeric insert.

In some embodiments, the filled polymer of a polymeric insert includes a different base polymer than that of the catheter component including the polymeric insert.

In some embodiments, the polymeric insert is overmolded in the catheter component.

In some embodiments, the polymeric insert is inserted into the catheter component.

In some embodiments, an antimicrobial filler of the filled polymer includes a carrier of the one-or-more antimicrobial metal species.

In some embodiments, the antimicrobial filler is about 8-15% by weight of each polymeric insert of the one-or-more polymeric inserts.

In some embodiments, the carrier is a zeolite and the one-or-more antimicrobial metal species include antimicrobial cations selected from silver, copper, and zinc electrostatically held at ion-exchange sites of the zeolite.

In some embodiments, the liquid includes dissolved cations selected from sodium and potassium for ion exchange with the antimicrobial cations at the ion-exchange sites of the zeolite.

Also disclosed herein is an antimicrobial catheter including, in some embodiments, one or more polymeric inserts of a filled polymer. Each polymeric insert of the one-or-more polymeric inserts provides at least a portion of a luminal surface in a catheter component selected from a catheter tube, a catheter hub, one or more extension legs, and one or more extension-leg fittings. Further, each polymeric insert of the one-or-more polymeric inserts is configured to elute one or more antimicrobial metal cations therefrom upon contact with a liquid including dissolved cations.

In some embodiments, the polymeric insert is overmolded in the catheter component or inserted into the catheter component.

In some embodiments, an antimicrobial filler of the filled polymer includes a carrier of the one-or-more antimicrobial metal cations. The carrier is a zeolite, and the one-or-more antimicrobial cations is selected from silver, copper, and zinc electrostatically held at ion-exchange sites of the zeolite for ion exchange with sodium, potassium, or a combination thereof for the dissolved cations.

Also disclosed herein is a method of making antimicrobial catheters. The method includes, in some embodiments, a polymeric insert-molding operation, a catheter component-molding operation, and a catheter-assembling operation. The polymeric insert-molding operation includes molding a plurality of polymeric inserts of a filled polymer. Each polymeric insert of the plurality of polymeric inserts is configured to elute one or more antimicrobial metal species therefrom upon contact with a liquid. The catheter component-molding operation includes molding a plurality of at least one catheter component selected from catheter components including a catheter tube, a catheter hub, an extension leg, and an extension-leg fitting. The catheter component includes one or more polymeric inserts providing at least a portion of a luminal surface in the catheter component. The catheter-assembling operation includes assembling the antimicrobial catheters from the catheter components.

In some embodiments, the method further includes a compounding operation. The compounding operation includes compounding a base polymer with an antimicrobial filler to form the filled polymer. The antimicrobial filler is about 8-15% by weight of the filled polymer.

In some embodiments, the antimicrobial filler includes a zeolite. Further, the one-or-more antimicrobial metal species include antimicrobial cations selected from silver, copper, and zinc electrostatically held at ion-exchange sites of the zeolite.

In some embodiments, the catheter component-molding operation includes overmolding the one-or-more polymeric inserts in the catheter component.

In some embodiments, the method further includes a polymeric insert-inserting operation. The polymeric insert-inserting operation includes inserting the one-or-more polymeric inserts in the catheter component after the catheter component-molding operation.

These and other features of the concepts provided herein will become more apparent to those of skill in the art in view of the accompanying drawings and following description, which describe particular embodiments of such concepts in greater detail.

### DRAWINGS

FIG. 1 illustrates an antimicrobial catheter in accordance with some embodiments.

FIG. 2 illustrates a polymeric insert providing at least a portion of a luminal surface in an extension-leg fitting in accordance with some embodiments.

FIG. 3 illustrates loading the polymeric insert on a core pin before molding the extension-leg fitting over the polymeric insert in accordance with some embodiments.

FIG. 4 illustrates molding the extension-leg fitting over the polymeric insert in accordance with some embodiments.

FIG. 5 illustrates unloading the extension-leg fitting from the core pin after molding the extension-leg fitting over the polymeric insert in accordance with some embodiments.

FIG. 6 illustrates elution of one or more antimicrobial metal species from a carrier thereof by way of exchange with exchange species in a liquid in contact with the carrier accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

As set forth above, CRBSIs are among the most frequent infections acquired in hospitals. The incidence of CRBSIs varies considerably depending on type of catheter, insertion site, clinician experience in catheter placement, duration of catheter placement, frequency of catheter access, and the like. While there has been a remarkable increase in epidemiological knowledge of CRBSIs as well as methodologies for diagnosis, management, and prevention thereof, CRBSIs still contribute to significant morbidity as well as lengthy and expensive hospital stays. Set forth below are antimicrobial catheters and methods that address the foregoing.

### Antimicrobial Catheters

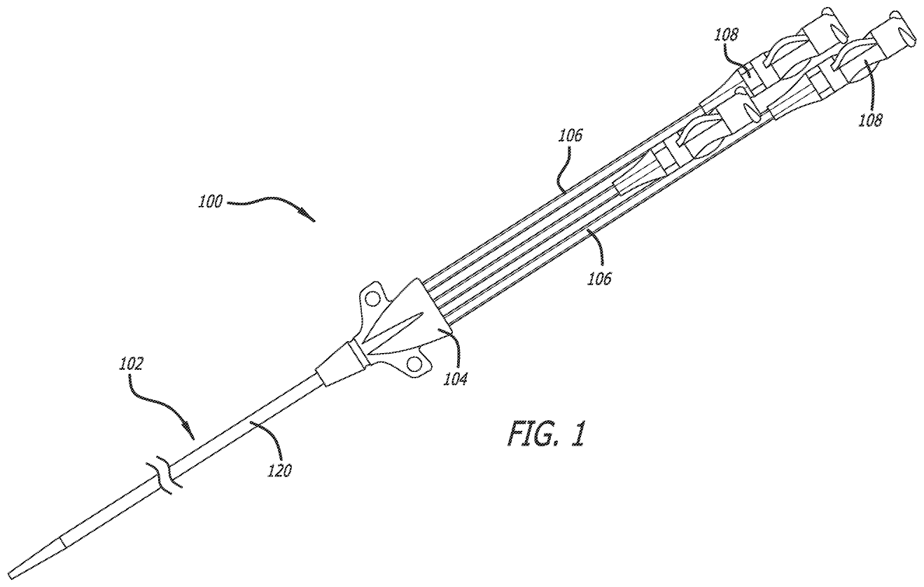

FIG. 1 illustrates an antimicrobial catheter 100 in accordance with some embodiments.

As shown, the antimicrobial catheter 100 can include one or more catheter components selected from a catheter tube 102, a catheter hub 104, one or more extension legs 106, and one or more extension-leg fittings 108 (e.g., connectors such as Luer connectors). When all of the foregoing catheter components are present in the antimicrobial catheter 100, as in FIG. 1, a proximal-end portion of the catheter tube 102 can be coupled to the catheter hub 104, a distal-end portion of each extension leg 106 of the one-or-more extension legs 106 can be coupled to the catheter hub 104, and a proximal-end portion of each extension leg 106 of the one-or-more extension legs 106 can be coupled to an extension-leg fitting 108 of the one-or-more extension-leg fittings 108. Such an antimicrobial catheter 100 can be a central venous catheter (“CVC”), a peripherally inserted central catheter (“PICC”), a midline catheter, a peripheral intravenous catheter (“PIVC”), an acute dialysis catheter, or the like; however the antimicrobial catheter 100 is not limited to such catheters.

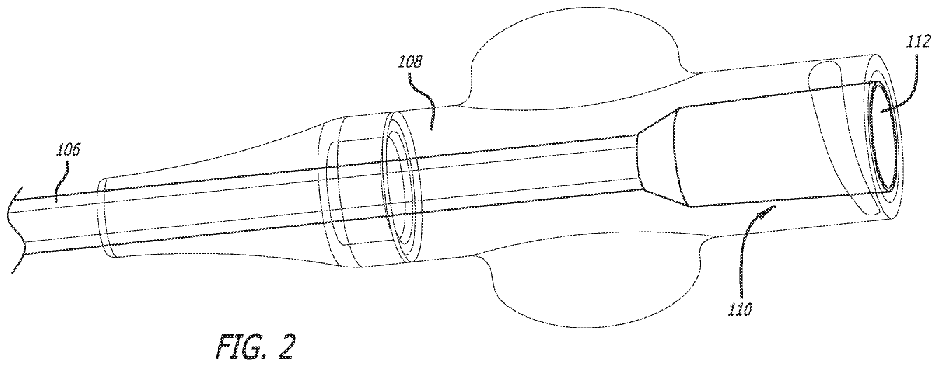

FIG. 2 illustrates a polymeric insert 110 providing a portion of a luminal surface 112 in accordance with some embodiments.

As shown, the antimicrobial catheter 100 can also include one or more polymeric inserts 110 of a filled polymer. Each polymeric insert 110 of the one-or-more polymeric inserts 110 can be overmolded or inserted into a catheter component selected from the catheter tube 102, the catheter hub 104, any extension leg(s) 106 of the one-or-more extension legs 106, and any extension-leg fitting(s) 108 of the one-or-more extension-leg fittings 108 such that the polymeric insert 110 provides at least a portion of the luminal surface 112 in its corresponding catheter component, such as a portion of the luminal surface 112 less than an entirety of the luminal surface 112 in its corresponding catheter component, for example, less than 75%, 50%, 25%, 15%, 10%, or 5% of the entirety of the luminal surface 112 in its corresponding catheter component. However, in some embodiments of the antimicrobial catheter 100, the polymeric insert 110 can provide up to the entirety of the luminal surface 112 of its corresponding catheter component, inclusive of the entirety of the luminal surface 112 of its corresponding catheter component.

In an example of the antimicrobial catheter 100, the catheter tube 102 can include at least one polymeric insert 110 of the one-or-more polymeric inserts 110 overmolded or inserted into the catheter tube 102. In another example of the antimicrobial catheter 100, the catheter hub 104 can include at least one polymeric insert 110 of the one-or-more polymeric inserts 110 overmolded or inserted into in a distal-end portion of the catheter hub 104; however, in a multiluminal catheter such as the triluminal antimicrobial catheter 100 shown in FIG. 1, the catheter hub 104 can include at least one polymeric insert 110 of the one-or-more polymeric inserts 110 overmolded or inserted into a proximal-end portion of the catheter hub 104 for each lumen of the multiluminal catheter. In another example of the antimicrobial catheter 100, each extension leg 106 of the one-or-more extension legs 106 can include at least one polymeric insert 110 of the one-or-more polymeric inserts 110 overmolded or inserted into the extension leg 106. In another example of the antimicrobial catheter 100, each extension-leg fitting 108 of the one-or-more extension-leg fittings 108 can include at least one polymeric insert 110 of the one-or-more polymeric inserts 110 overmolded or inserted into the extension-leg fitting 108 as shown in FIG. 2. Notably, the polymeric insert 110 overmolded or inserted into the extension-leg fitting 108 shown in FIG. 2 abuts a proximal end of the extension leg 106 also overmolded or inserted into the extension-leg fitting 108 such that the polymeric insert 110 provides a portion of the luminal surface 112 of the extension-leg fitting 108 less than the entirety of the luminal surface 112 of the extension-leg fitting 108, a remainder of the luminal surface 112 of the extension-leg fitting 108 being provided by the extension leg 106. Should a plurality of the catheter components of the antimicrobial catheter 100 include a plurality of the polymeric inserts 110, optionally, respectively, the plurality of polymeric inserts 110 can be independently overmolded or inserted into their corresponding catheter components.

The filled polymer of any polymeric insert 110 of the one-or-more polymeric inserts 110 can include a same or different base polymer than that of the catheter component including the polymeric insert 110. In an example, the filled polymer of the one-or-more polymeric inserts 110 can include a thermoplastic polyurethane as a base polymer, and the one-or-more polymeric inserts 110 can be respectively overmolded or inserted into the one-or-more extension-leg fittings 108 also having the thermoplastic polyurethane as the base polymer thereof. Whether the base polymer of the filled polymer is the same or different than that of any of the catheter components of the antimicrobial catheter 100, the filled polymer can include one or more additional polymers to the base polymer (e.g., a hydrophilic polymer to facilitate exchange of the one-or-more antimicrobial metal species 116 with the exchange species 118) for a polymer blend thereof, one or more additives selected from plasticizers, lubricants, impact modifiers, antistatic agents, and colorants, or a combination thereof.

Notably, the filled polymer includes at least an antimicrobial filler for a filler-type additive, the antimicrobial filler including a carrier 114 of one or more antimicrobial metal species 116 or, in some embodiments, the one-or-more antimicrobial metal species 116 exclusive of the carrier 114. The carrier 114 of the one-or-more antimicrobial metal species 116 can, in turn, be a mineral such as one or more zeolites, bentonite, montmorillonite, vermiculite, illite, hydroxyapatite or a resin such as ion-exchange resin or a chelating resin. Such a carrier 114 can electrostatically hold the one-or-more antimicrobial metal species 116 at exchange sites of the carrier 114, include the one-or-more antimicrobial metal species 116 in pores of the carrier 114, or a combination thereof. In an example, a zeolite can electrostatically hold the one-or-more antimicrobial metal species 116 at ion-exchange sites of the zeolite or include the one-or-more antimicrobial metal species 116 in pores of the zeolite, the one-or-more antimicrobial metal species 116 including antimicrobial cations selected from silver, copper, and zinc cations in some embodiments. Notably, such an antimicrobial filler can be an Agion® antimicrobial product such as Agion® silver, Agion® Lg, Agion® copper, Agion® zinc, or Agion® 3x by Sciessent, LLC (Beverly, MA), but it should be understood the antimicrobial filler is not limited to any one or more Agion® antimicrobial products. Indeed, as above, the antimicrobial filler can even be exclusive of the carrier 114, and, in such embodiments the one-or-more antimicrobial metal species 116 can be any one or more of the foregoing metal cations (e.g., silver cation), one or more metal nanoparticles (e.g., silver nanoparticles), etc.

The antimicrobial filler of the filled polymer of the one-or-more polymeric inserts 110 can be at least about 1% by weight of the filled polymer, including at least about 10% by weight of the filled polymer, such as at least about 20% by weight of the filled polymer, for example, at least about 30%, 40% or 50% by weight of the filled polymer, or an intervening concentration thereof (e.g., at least about 8% by weight, 15% by weight, 22% by weight of the filled polymer) as defined by the least significant digit in the foregoing concentrations. Alternatively, the antimicrobial filler can be no more than about 50% by weight of the filled polymer, including no more than about 40% by weight of the filled polymer, such as no more than about 30% by weight of the filled polymer, for example, no more than about 20%, 10% or 10% by weight of the filled polymer, or an intervening concentration thereof (e.g., no more than about 22% by weight, 15% by weight, 8% by weight of the filled polymer) as defined by the least significant digit in the foregoing concentrations. In an example, the antimicrobial filler can be about 8-15% by weight of the filled polymer such that the antimicrobial filler is correspondingly about 8-15% by weight of each polymeric insert 110 of the one-or-more polymeric inserts 110.

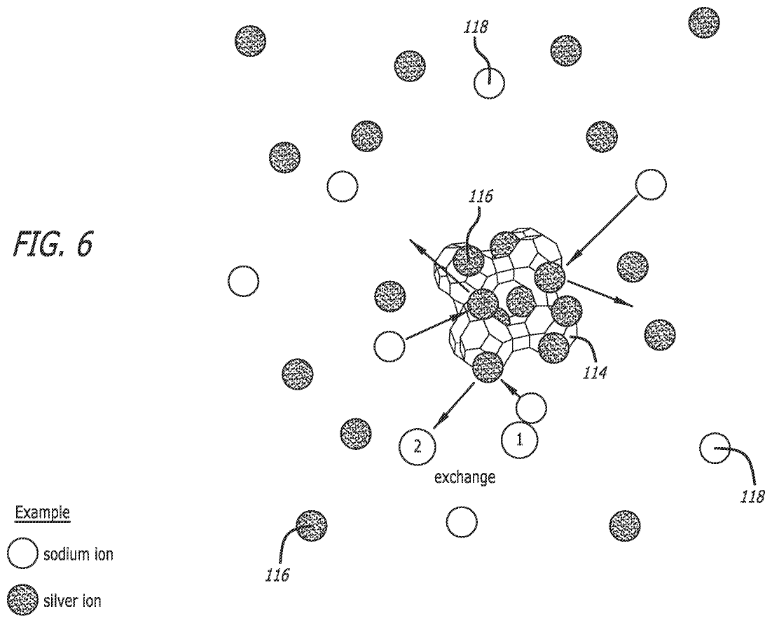

FIG. 6 illustrates elution of the one-or-more antimicrobial metal species 116 from the carrier 114 by way of exchange with exchange species 118 in a liquid in contact with the carrier 114 accordance with some embodiments.

As shown, each polymeric insert 110 of the one-or-more polymeric inserts 110 can be configured to elute the one-or-more antimicrobial metal species 116 from the carrier 114 in the filled polymer of the polymeric insert 110 upon contact with the liquid, thereby conferring antimicrobial activity to the antimicrobial catheter 100. In an example, each polymeric insert 110 of the one-or-more polymeric inserts 110 can be configured to elute the one-or-more antimicrobial metal cations (e.g., silver, copper, zinc, or a combination thereof) from the zeolite in the filled polymer of the polymeric insert 110 upon contact with the liquid, thereby conferring antimicrobial activity to the antimicrobial catheter 100. Such a liquid can be an aqueous mixture such as an aqueous suspension (e.g., blood), an aqueous solution (e.g., saline), or merely water-containing moisture including dissolved cations for the exchange species 118, the dissolved cations selected from at least sodium and potassium for ion exchange with the one-or-more antimicrobial cations at the ion-exchange sites of the zeolite.

Notably, the antimicrobial activity of the antimicrobial catheter 100 need not be limited to that conferred to the antimicrobial catheter 100 by the one-or-more polymeric inserts 110. In an example, the catheter tube 102 can include an antimicrobial coating on at least an abluminal surface 120 thereof. In another example, an entirety of the catheter tube 102, the catheter hub 104, the one-or-more extension legs 106, the one-or-more extension-leg fittings 108, or the antimicrobial catheter 100 assembled from such catheter components can include another antimicrobial filler (e.g., silver acetate) compounded into the base polymer(s) thereof different than that of the filled polymer of the one-or-more polymeric inserts 110. In particular, the other antimicrobial filler compounded into the base polymer(s) of the catheter tube 102, the catheter hub 104, the one-or-more extension legs 106, the one-or-more extension-leg fittings 108, or the antimicrobial catheter 100 assembled therefrom should not be any one or more Agion® antimicrobial products set forth above.

Indeed, it has been found that only about 1% or less of the one-or-more antimicrobial metal species 116 are released from such catheter components or antimicrobial catheters (e.g., released from luminal surfaces 112 of the catheter components or antimicrobial catheters) under typical usage conditions when the catheter components or antimicrobial catheters assembled therefrom include about 8-15% by weight of an antimicrobial filler such as an Agion® antimicrobial product compounded into the base polymer(s) thereof. The remaining 99% or so of the one-or-more antimicrobial metal species 116 remain trapped in bodies of the foregoing catheter components or antimicrobial catheters under such usage conditions. Further, it has been found that the foregoing catheter components or antimicrobial catheters assembled therefrom require special manufacturing considerations such as extensive drying (e.g., multiday vacuum drying) of the filled polymer therefor due to the 8-15%-by-weight concentration of the foregoing antimicrobial filler. In contrast, the one-or-more polymeric inserts 110 overmolded or inserted into the catheter tube 102, the catheter hub 104, the one-or-more extension legs 106, or the one-or-more extension-leg fittings 108 provide the antimicrobial activity precisely where it is needed in the antimicrobial catheter 100, reducing manufacturing costs associated with unusable or unavailable antimicrobial filler and obviating manufacturing considerations such as extensive drying, importantly, without sacrificing antimicrobial efficacy.

Further, the antimicrobial activity conferred to the antimicrobial catheter 100 by the one-or-more polymeric inserts 110 is advantageous in its on-demand nature upon contact with the liquid set forth above. Indeed, the on-demand nature of the antimicrobial activity conferred to the antimicrobial catheter 100 by the one-or-more polymeric inserts 110 contributes to maintaining the antimicrobial efficacy of the antimicrobial catheter 100 over an extended period of time such as from packaging the antimicrobial catheter 100, through storing the antimicrobial catheter 100 by a supplier and later, a clinical facility, and to using the antimicrobial catheter 100. While one or more metal inserts (e.g., one or more copper inserts) overmolded or inserted into the catheter tube 102, the catheter hub 104, the one-or-more extension legs 106, or the one-or-more extension-leg fittings 108 might alternatively provide a sufficient level of antimicrobial activity, the antimicrobial activity of the one-or-more metal inserts is, in contrast, of an always-on nature due to the antimicrobial activity being dependent on exposure of a luminal surface of each metal insert of the one-or-more metal inserts. Such luminal-surface exposure diminishes antimicrobial efficacy of the one-or-more metal inserts over time (e.g., from packaging, through storing, and to using a catheter including the one-or-more metal inserts) due to surface oxidation, which is clearly a disadvantage compared to the one-or-more on-demand polymeric inserts 110. Notwithstanding the foregoing disadvantage, the one-or-more metal inserts (e.g., one or more silver, copper, or zinc inserts) can additionally or alternatively be overmolded or inserted into the catheter tube 102, the catheter hub 104, the one-or-more extension legs 106, or the one-or-more extension-leg fittings 108 to meet a specific need of the antimicrobial catheter 100 that outweighs such a disadvantage.

### Methods

Methods include methods of making antimicrobial catheters such as the antimicrobial catheter 100 as well as methods of using the antimicrobial catheter 100.

As to a method of making antimicrobial catheters, such as method can include one or more operations selected from a compounding operation, a polymeric insert-molding operation, a catheter component-molding operation, a polymeric insert-inserting operation, and a catheter-assembling operation.

The compounding operation can include compounding the base polymer for a plurality of polymeric inserts 110 with the antimicrobial filler to form the filled polymer for the plurality of polymeric inserts 110. Such a compounding operation can include heating the base polymer for a softened or melted base polymer; gradually or continuously adding the antimicrobial filler to the softened or melted base polymer; mixing the softened or melted base polymer with the antimicrobial filler in a kneader, mixer or the like until the antimicrobial filler is homogeneously dispersed throughout the softened or melted base polymer for a softened or melted filled polymer; cooling the softened or melted filled polymer for a solidified filled polymer; and, optionally, pelletizing the solidified filled polymer or the like for ease of handling the filled polymer during the polymeric insert-molding operation. As set forth above, the antimicrobial filler can include the zeolite with the one-or-more antimicrobial metal species 116 being antimicrobial cations selected from silver, copper, and zinc electrostatically held at the ion-exchange sites of the zeolite, disposed in the pores of the zeolite, or a combination thereof. Further, the antimicrobial filler can be about 8-15% by weight of the filled polymer for about 8-15% by weight of each polymeric insert 110 of the plurality of polymeric inserts 110 after the polymeric insert-molding operation.

The polymeric insert-molding operation can include molding (e.g., blow molding, injection molding, etc.) the plurality of polymeric inserts 110 of the filled polymer. Each polymeric insert 110 of the plurality of polymeric inserts 110 can be configured to elute the one-or-more antimicrobial metal species 116 therefrom upon contact with the liquid set forth above.

The catheter component-molding operation can include molding (e.g., injection molding, compression molding, extrusion molding, etc.) a plurality of at least one catheter component selected from the catheter components including the catheter tube 102, the catheter hub 104, an extension leg 106 of the one-or-more extension legs 106 set forth above, and an extension-leg fitting 108 of the one-or-more extension-leg fittings 108 set forth above. The catheter component can include the one-or-more polymeric inserts 110 providing a portion of the luminal surface 112 less than an entirety of the luminal surface 112 in the catheter component. However, as noted above, the one-or-more polymeric inserts 110 can provide up to an entirety of the luminal surface 112 in the catheter component in some embodiments.

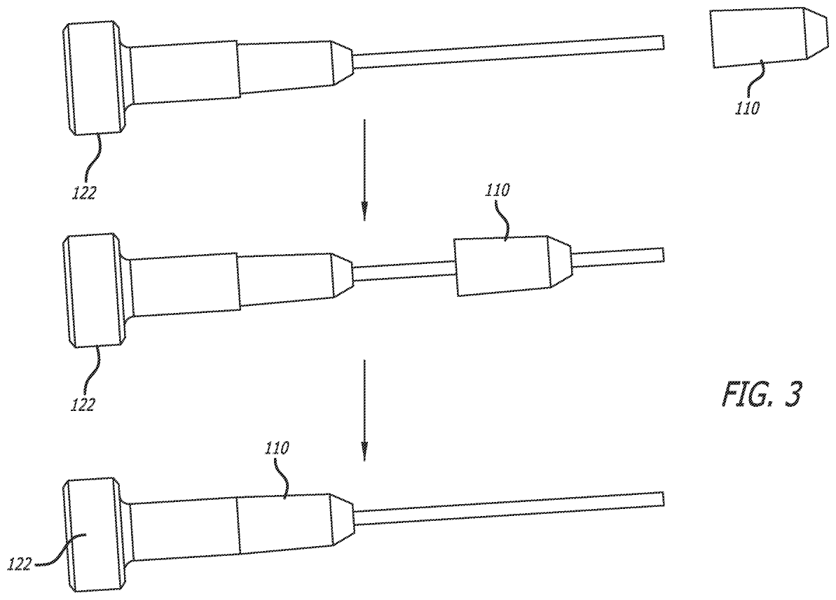

FIGS. 3-5 illustrate the catheter component-molding operation for molding (e.g., injection molding) a plurality of extension-leg fittings 108 over a respective plurality of polymeric inserts 110 in accordance with some embodiments.

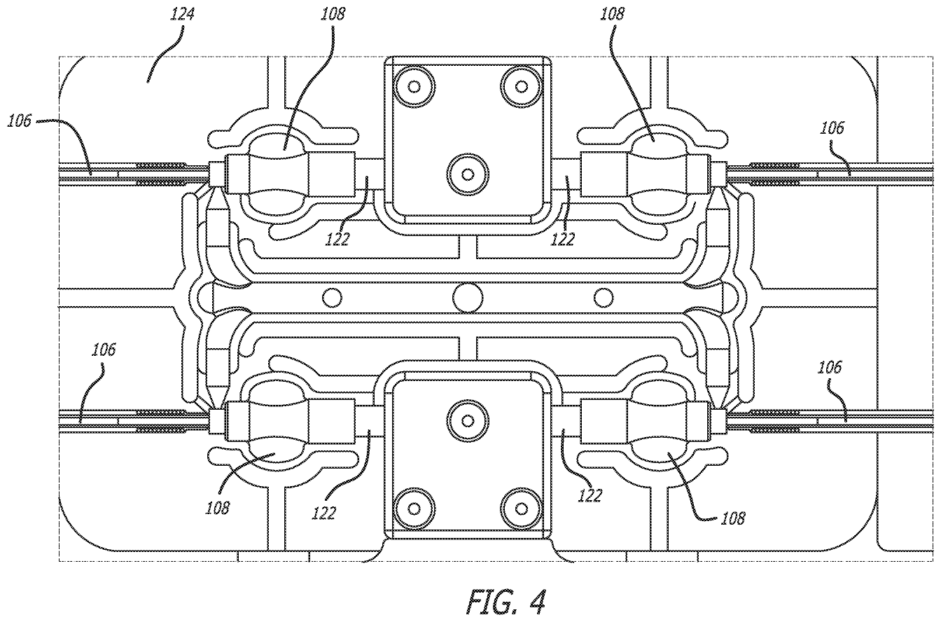

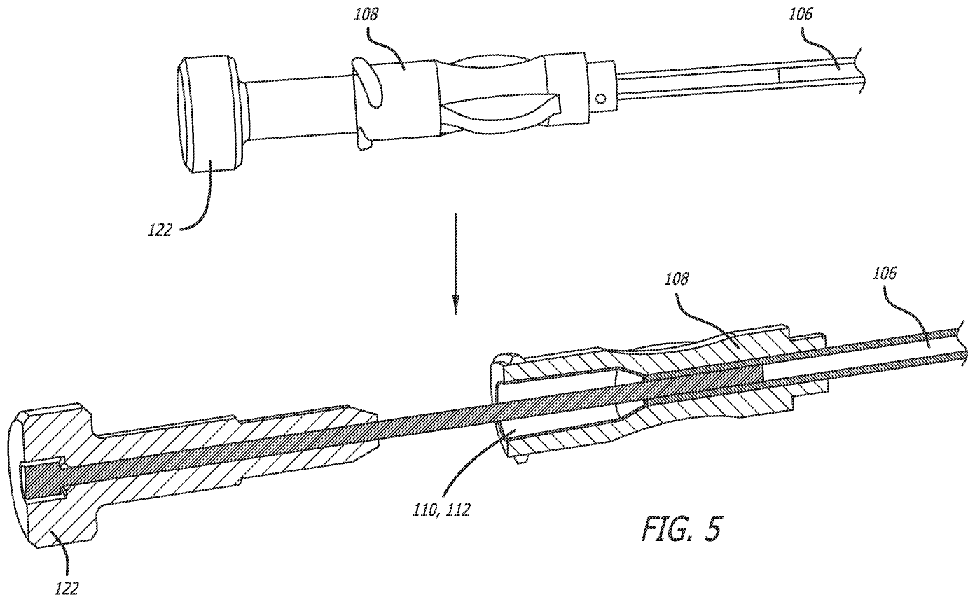

In an example of the catheter component-molding operation, the catheter component-molding operation can include loading each polymeric insert 110 of the plurality of polymeric inserts 110 on a core pin 122, as shown in FIG. 3. Further, each core pin 122 of a plurality of core pins 122 respectively including the plurality of polymeric inserts 110 can be disposed in a mold 124, and the plurality of extension-leg fittings 108 can be respectively injection molded over the plurality of polymeric inserts 110, as shown in FIG. 4. Lastly, as shown in FIG. 5, the plurality of core pins 122 respectively including the plurality of extension-leg fittings 108 molded over the plurality of polymeric inserts 110 can be removed from the mold 124, and each extension-leg fitting 108 of the plurality of extension-leg fittings 108 can be unloaded from its corresponding core pin 122 of the plurality of core pins 122 for the catheter-assembling operation.

The polymeric insert-inserting operation can include inserting the one-or-more polymeric inserts 110 in the catheter component after the catheter component-molding operation if the catheter component-molding operation does not include overmolding the one-or-more polymeric inserts 110 in the catheter component. While the one-or-more polymeric inserts 110 can be mechanically coupled to the catheter component by way of one or more interference fits in accordance with the inserting of the one-or-more polymeric inserts 110 in the catheter component, thermal bonding, solvent bonding, or adhesive bonding can alternatively or additionally be used.

The catheter-assembling operation can include assembling the antimicrobial catheters from the catheter components such that in each antimicrobial catheter 100 the proximal-end portion of the catheter tube 102 is coupled to the catheter hub 104, the distal-end portion of each extension leg 106 of the one-or-more extension legs 106 is coupled to the catheter hub 104, and the proximal-end portion of each extension leg 106 of the one-or-more extension legs 106 is coupled to an extension-leg fitting 108 of the one-or-more extension-leg fittings 108. Notably, the catheter-assembling operation need not involve inserting the proximal-end portion of the catheter tube 102 into the catheter hub 104, the distal-end portion of each extension leg 106 of the one-or-more extension legs 106 into the catheter hub 104, or the proximal-end portion of each extension leg 106 of the one-or-more extension legs 106 into an extension-leg fitting 108 of the one-or-more extension-leg fittings 108 if such couplings are effectuated during the catheter component-molding operation. In an example, the proximal-end portion of each extension leg 106 of the one-or-more extension legs 106 shown in FIG. 4 is coupled to an extension-leg fitting 108 of the one-or-more extension-leg fittings 108 by overmolding during the catheter component-molding operation.

As to a method of using the antimicrobial catheter 100, such as method can include one or more operations selected from a catheter-placing operation, a catheter-securing operation, and a catheter-permitting operation. The catheter-placing operation can include placing the antimicrobial catheter 100 in a vasculature of a patient such as by the Seldinger technique. The catheter-securing operation can include securing the antimicrobial catheter 100 to the patient such as by a catheter securement device, wound dressing, or a combination thereof. The catheter-permitting operation can include permitting the antimicrobial catheter 100 to elute one or more antimicrobial metal species 116 therefrom in accordance with its contact with a liquid (e.g., a body fluid such as blood, saline, infusate, etc.), as set forth above.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An antimicrobial catheter, comprising:
a catheter tube;
a catheter hub, a proximal-end portion of the catheter tube coupled to the catheter hub;
one or more extension legs, a distal-end portion of each extension leg of the one-or-more extension legs coupled to the catheter hub;
one or more extension-leg fittings, a proximal-end portion of each extension leg of the one-or-more extension legs coupled to an extension-leg fitting of the one-or-more extension-leg fittings; and
one or more polymeric inserts of a filled polymer, each polymeric insert of the one-or-more polymeric inserts providing at least a portion of a luminal surface in a catheter component selected from the catheter tube, the catheter hub, the one-or-more extension legs, and the one-or-more extension-leg fittings, and each polymeric insert of the one-or-more polymeric inserts configured to elute one or more antimicrobial metal species therefrom upon contact with a liquid.

2. The antimicrobial catheter of claim 1, wherein the catheter tube includes at least one polymeric insert of the one-or-more polymeric inserts.

3. The antimicrobial catheter of claim 1, wherein the catheter tube includes an antimicrobial compounded into the catheter tube different than the one-or-more antimicrobial metal species of the one-or-more polymeric inserts.

4. The antimicrobial catheter of claim 1, wherein the catheter hub includes at least one polymeric insert of the one-or-more polymeric inserts.

5. The antimicrobial catheter of claim 1, wherein each extension leg of the one-or-more extension legs includes at least one polymeric insert of the one-or-more polymeric inserts.

6. The antimicrobial catheter of claim 1, wherein each extension-leg fitting of the one-or-more extension-leg fittings includes at least one polymeric insert of the one-or-more polymeric inserts.

7. The antimicrobial catheter of claim 1, wherein the filled polymer of a polymeric insert includes a same base polymer as that of the catheter component including the polymeric insert.

8. The antimicrobial catheter of claim 1, wherein the filled polymer of a polymeric insert includes a different base polymer than that of the catheter component including the polymeric insert.

9. The antimicrobial catheter of claim 7, wherein the polymeric insert is overmolded in the catheter component.

10. The antimicrobial catheter of claim 7, wherein the polymeric insert is inserted into the catheter component.

11. The antimicrobial catheter of claim 1, wherein an antimicrobial filler of the filled polymer includes a carrier of the one-or-more antimicrobial metal species.

12. The antimicrobial catheter of claim 11, wherein the antimicrobial filler is about 8-15% by weight of each polymeric insert of the one-or-more polymeric inserts.

13. The antimicrobial catheter of claim 11, wherein the carrier is a zeolite and the one-or-more antimicrobial metal species include antimicrobial cations selected from silver, copper, and zinc electrostatically held at ion-exchange sites of the zeolite.

14. The antimicrobial catheter of claim 13, wherein the liquid includes dissolved cations selected from sodium and potassium for ion exchange with the antimicrobial cations at the ion-exchange sites of the zeolite.

15. An antimicrobial catheter, comprising:
one or more polymeric inserts of a filled polymer, each polymeric insert of the one-or-more polymeric inserts providing at least a portion of a luminal surface in a catheter component selected from a catheter tube, a catheter hub, one or more extension legs, and one or more extension-leg fittings, and each polymeric insert of the one-or-more polymeric inserts configured to elute one or more antimicrobial metal cations therefrom upon contact with a liquid including dissolved cations.

16. The antimicrobial catheter of claim 15, wherein the polymeric insert is overmolded in the catheter component or inserted into the catheter component.

17. The antimicrobial catheter of claim 15, wherein an antimicrobial filler of the filled polymer includes a carrier of the one-or-more antimicrobial metal cations, the carrier being a zeolite and the one-or-more antimicrobial cations selected from silver, copper, and zinc electrostatically held at ion-exchange sites of the zeolite for ion exchange with sodium, potassium, or a combination thereof for the dissolved cations.

18. A method of making antimicrobial catheters, comprising:
molding a plurality of polymeric inserts of a filled polymer, each polymeric insert of the plurality of polymeric inserts configured to elute one or more antimicrobial metal species therefrom upon contact with a liquid;
molding a plurality of at least one catheter component selected from catheter components including a catheter tube, a catheter hub, an extension leg, and an extension-leg fitting, the catheter component including one or more polymeric inserts providing at least a portion of a luminal surface in the catheter component; and
assembling the antimicrobial catheters from the catheter components.

19. The method of claim 18, further comprising compounding a base polymer with an antimicrobial filler to form the filled polymer, the antimicrobial filler being about 8-15% by weight of the filled polymer.

20. The method of claim 19, wherein the antimicrobial filler includes a zeolite, the one-or-more antimicrobial metal species including antimicrobial cations selected from silver, copper, and zinc electrostatically held at ion-exchange sites of the zeolite.

21. The method of claim 18, wherein the molding of the plurality of the at-least-one catheter component includes overmolding the one-or-more polymeric inserts in the catheter component.

22. The method of claim 18, further comprising inserting the one-or-more polymeric inserts in the catheter component after the molding of the plurality of the at-least-one catheter component.
